(19) United States
(12) Patent Application Publication
(10) Pub. No.: US 2003/0211482 A1
(43) Pub. Date: Nov. 13, 2003

(54) Method for nucleic acid detection

(76) Inventor: Tzong Hae Lee, San Francisco, CA (US)

Correspondence Address: STEVENS, DAVIS, MILLER & MOSHER, L.L.P., 1615 L STREET, N.W., Suite 850, WASHINGTON, DC, 20036, US

(21) Appl. No.: 10/142,955

(22) Filed: May 13, 2002

(51) Int. Cl. C12Q 1/68; C12P 19/34

(52) U.S. Cl. 435/6; 435/91.2

(57) ABSTRACT

A method for nucleic acid detection includes the steps of (a) adding to the sample a thermostable polymerase, appropriate nucleoside triphosphates, a nucleic-acid-binding fluorescent entity, and a pair of first and second primers that have nucleotide sequences substantially complementary to a DNA which flanks the target nucleic acid wherein the 3′-end nucleotide of the first primer is complementary to at least one variant nucleotide in a target nucleic acid contained in a sample; (b) thermally cycling the sample between at least a denaturation temperature and an elongation temperature; (c) illuminating the sample with a selected wavelength of light that is absorbed by the fluorescent entity during the thermally cycling step; (d) monitoring an amplification dependent emission of the fluorescent entity; and (e) detecting the presence or absence of the variant nucleotide by judging the presence or absence of an amplified product of the DNA.

### BACKGROUND OF THE INVENTION

1. Field of the Invention

The invention relates to nucleic acid detection. More specifically, the invention relates to the determination of the presence or absence of one or more variant nucleotides in a target nucleic acid.

2. Description of the Related Art

Methods to detect nucleic acids provide a foundation upon which the large and rapidly growing field of molecular biology is built. In the post-genome era, sensitive and reliable methods for single nucleotide polymorphism (SNP) assay and gene expression profiling are urgently needed. A variety of methodologies currently exist for the detection of single nucleotide polymorphisms (SNPs) that are present in genomic DNA. SNPs are DNA point mutations or insertions/deletions that are present at measurable frequencies in the population. SNPs are the most common variations in the genome. SNPs occur at defined positions within genomes and can be used for gene mapping, defining population structure, and performing functional studies. SNPs are useful as markers because many known genetic diseases are caused by point mutations and insertions/deletions.

In rare cases where an SNP alters a restriction enzyme recognition sequence, differential sensitivity of the amplified DNA to cleavage can be used for SNP detection. This technique requires that an appropriate restriction enzyme site be present or introduced in the appropriate sequence context for differential recognition by the restriction endonuclease. After amplification, the products are cleaved by the appropriate restriction endonuclease and products are analyzed by gel electrophoresis, followed by Southern blotting, hybridization and detection of radioactively labeled probe. The throughput of analysis by this technique is limited because samples require processing, gel analysis, and significant interpretation of data before SNPs can be accurately determined.

Single strand conformational polymorphism (SSCP) is another well-known technique that can detect SNPs present in an amplified DNA segment. In this method, the double stranded amplified product is denatured and then both strands are allowed to reanneal during electrophoresis in non-denaturing polyacrylamide gels. However, the design and interpretation of SSCP based experiments can be difficult and the throughput of gel-based techniques is quite limited.

In addition, single base changes can be detected by gel electrophoresis following a primer extension reaction. In this technique, a region of genomic DNA containing the SNP of interest is amplified. Following the PCR amplification, a SNP primer first anneals to the PCR product such that the 3′ end of the primer is adjacent to the location of the SNP, and then the primer is extended by a single dideoxynucleotide (ddNTP). After the primer extension reaction is complete, electrospray mass spectrometry (ESI/MS) is used to quantitate the ddNTPs remaining in the reaction solution. If one or more bases were incorporated into the SNP primer, then the signal intensity from these bases will be reduced in the mass spectrum of the reaction solution. The SNP bases can be identified by comparing the peak area or the peak height of the four ddNTPs in test samples to those of a control sample. This technique is sensitive and reliable but relatively inefficient.

Therefore, there is a need for another method to determine the presence or absence of a target nucleic acid in a nucleic acid sample.

### SUMMARY OF THE INVENTION

It is an object of the present invention to provide a simple method for directly detecting at least one single base difference in nucleic acids such as genomic DNA in which detection steps are minimized resulting in a method which may be performed quickly, accurately and easily with minimal operator skill.

To achieve the above listed and other objects, the method of the present invention comprises the steps of (a) adding to the sample a thermostable polymerase, appropriate nucleoside triphosphates, a nucleic-acid-binding fluorescent entity, and a pair of first and second primers that have nucleotide sequences substantially complementary to a DNA which flanks the target nucleic acid wherein the 3′-end nucleotide of the first primer is complementary to at least one variant nucleotide in a target nucleic acid contained in a sample; (b) thermally cycling the sample between at least a denaturation temperature and an elongation temperature; (c) illuminating the sample with a selected wavelength of light that is absorbed by the fluorescent entity during the thermally cycling step; (d) monitoring an amplification dependent emission of the fluorescent entity; and (e) detecting the presence or absence of the variant nucleotide by judging the presence or absence of an amplified product of the DNA.

The present invention is based on the fact that by selecting the 3′-end nucleotide of the first primer complementary to the variant nucleotide of a mutated form of genomic DNA, the amplified product of the DNA flanking the target nucleic acid is only synthesized if the sample contains the mutated form of the target nucleic acid. Therefore, the wild-type can easily be discriminated from the mutant type by judging the presence or absence of the amplified product of the DNA flanking the target nucleic acid.

Nucleic-acid-binding fluorescent entity, e.g., a double strand specific nucleic acid binding dye or a fluorescently labeled oligonucleotide probe, is used for the detection and analysis of the amplified product of the DNA flanking the target nucleic acid without the need for any subsequent handling step, thereby allowing a high-through-put SNP detection.

In another embodiment of the present invention, the 3′-end nucleotide of the second primer is complementary to the 3′-end nucleotide of the first primer. This may enable discrimination and specificity to be increased since any non-specific product requires priming to occur at the relevant 3′-end of two mismatched primers.

According to a further feature of the present invention there is provided a kit for detecting the presence or absence of at least one variant nucleotide in a target nucleic acid contained in a sample. The kit comprises a pair of first and second primers that have nucleotide sequences substantially complementary to a DNA which flanks the target nucleic acid wherein the 3′-end nucleotide of the first primer is complementary to the variant nucleotide, four different nucleoside triphosphates, a nucleic-acid-binding fluorescent entity, and a thermostable polymerase.

### BRIEF DESCRIPTION OF THE DRAWINGS

Other objects, advantages, and novel features of the invention will become more apparent from the following detailed description when taken in conjunction with the accompanying drawings:

FIG. 1a and FIG. 1b illustrate one embodiment of the invention;

FIG. 2 shows the results obtained according to the method illustrated in FIG. 1 by plotting fluorescence signal versus cycle number; and

FIG. 3 illustrates another embodiment of the present invention.

### DETAILED DESCRIPTION OF THE PREFERRED EMBODIMENT

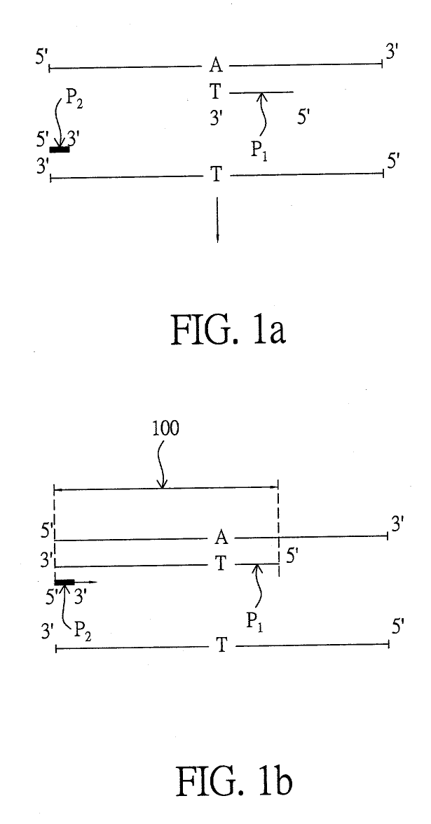

This invention provides a high-through-put method for detecting the presence or absence of at least one variant nucleotide in a target nucleic acid contained in a sample. According to one embodiment of the present invention, the target nucleic acid may contain a G→A point mutation site, for example as a result of a genetic disorder. FIG. 1a shows denatured genomic DNA strands contain the variant nucleotide, i.e., the mutant nucleotide (A). The region of the genomic DNA subject to amplification is designated 100 in FIG. 1b.

First, the method of the present invention is performed by adding to the sample a thermostable polymerase, appropriate nucleoside triphosphates, a nucleic-acid-binding fluorescent entity, and a pair of primers P1/P2 (see FIG. 1(b)) to create an amplification medium. The primers have nucleotide sequences substantially complementary to the DNA 100 (see FIG. 1(b)) which flanks the target nucleic acid. The primer P1 must anneal immediately upstream of the mutation site to be detected. It should be understood that the primer P1 can be chosen on either the sense or antisense strand, as long as the 3′-end anneals to at least one variant nucleotide in the mutation site. It is noted that the primer P1 has a 3′-end nucleotide (T) complementary to the mutant nucleotide (A). The term “nucleoside triphosphate” is used herein to refer to nucleosides present in either DNA or RNA and thus includes nucleosides which incorporate adenine, cytosine, guanine, thymine and uracil as base, the sugar moiety being deoxyribose or ribose. Suitable nucleic-acid-binding fluorescent entity for detecting and monitoring DNA amplification include double strand specific nucleic acid binding dyes or fluorescently labeled oligonucleotide probes. Those skilled in the art will be familiar with the use of ethidium bromide in monitoring DNA amplification. When a double strand-specific fluorescent dye is present during amplification, fluorescence generally increases as more double stranded product is made. It is preferred that SYBR® Green I, which is well known in the art and available from Molecular Probes of Eugene, Oreg., be used as a double-strand-specific dye. The molecular structure of this dye is a trade secret, but it is recommended by the manufacturer as a more sensitive double-strand-specific dye for DNA detection. A suitable fluorescently labeled probe is an oligonucleotide with both a reporter fluorescent dye and a quencher dye attached. While the probe is intact, the proximity of the quencher greatly reduces the fluorescence emitted by the reporter dye by Forster resonance energy transfer (FRET) through space.

Thereafter, the amplification medium is placed in a thermocycler for performing a thermally cycling reaction between at least a denaturation temperature and an elongation temperature. A suitable thermocycler is an instrument designed to detect fluorescence during the thermal cycling reaction, e.g., GeneAmp® 5700 Sequence Detection System. During the elongation stage of the thermally cycling reaction, chain extension of the primer P1 containing the nucleotide (T) in the 3′-end is observed (see FIG. 1b). It is noted that no such chain extension from the primer P1 when the sample contains only the wild-type nucleic acid.

Finally, the amplification medium is irradiated with a selected wavelength of light and the resulting fluorescence is detected using a CCD array. Fluorescence values are recorded during every thermal cycle and represent the amount of product amplified to that point in the amplification reaction. Software built in thermocycler collects the images throughout the thermal cycling of PCR and analyzes the data to generate an amplification plot for each sample by plotting fluorescence signal versus cycle number.

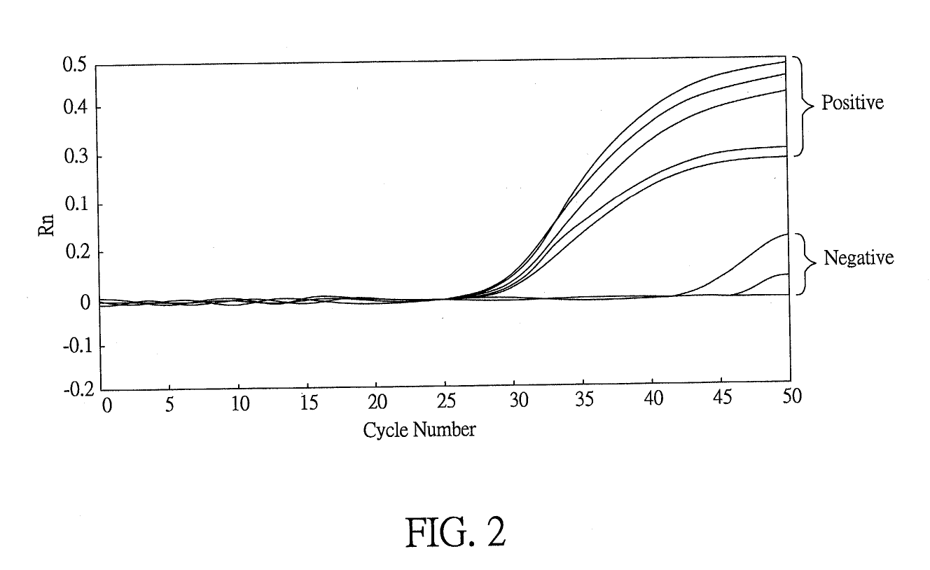

The results of effecting the method illustrated in FIG. 1 is shown by plotting fluorescence signal of different samples versus cycle number. Typically, the more template containing the mutated form of the target nucleic acid present at the beginning of the amplification reaction, the fewer number of cycles it takes to reach a point in which the fluorescent signal is first recorded as statistically significant above background. This point is defined as the CT (threshold cycle), and will always occur during the exponential phase of amplification. Therefore, if the sample contains the mutated form of the target nucleic acid, the amplification plot will be similar to those designated “Positive” in FIG. 2. Since the amplified product of the DNA 100 flanking the target nucleic acid is only synthesized if the sample contains the mutated form of the target nucleic acid, the mutant type can easily be discriminated from the wild-type by determining if the calculated CT of a sample reaction is above a predetermined value.

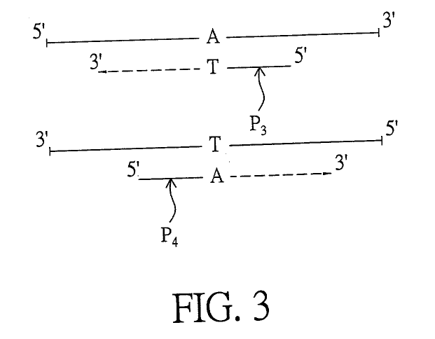

FIG. 3 illustrates another embodiment of the present invention. Two primers P3/P4 are employed for each strand of a double stranded nucleic acid. It is noted that the primer P3 has a 3′-end nucleotide (T) complementary to the mutant nucleotide (A) and the primer P4 has a 3′-end nucleotide (A) complementary to the 3′-end nucleotide (T) of the primer P3. The method thus substantially proceeds as described in relation to FIG. 1 but with increased specificity since any non-specific product requires priming to occur at the relevant 3′-end of the primers P3/P4.

It should be appreciated that while the method of the present invention is of particular interest in detecting the presence or absence of point mutations, the method is equally applicable to detecting the presence or absence of deletions, including deletions of more than one nucleotide as well as to detecting the presence or absence of substitutions of more than one nucleotide. In this regard it is simply necessary to know the relevant nucleotides, especially the relevant terminal nucleotide, so that the necessary primer(s) may be designed appropriately.

Although the invention has been explained in relation to its preferred embodiment, it is to be understood that many other possible modifications and variations can be made without departing from the spirit and scope of the invention as hereinafter claimed.

What is claimed is:

1. A method for detecting the presence or absence of at least one variant nucleotide in a target nucleic acid contained in a sample, the method comprising the steps of:
adding to the sample a thermostable polymerase, appropriate nucleoside triphosphates, a nucleic-acid-binding fluorescent entity, and a pair of first and second primers that have nucleotide sequences substantially complementary to a DNA which flanks the target nucleic acid wherein the 3′-end nucleotide of the first primer is complementary to the variant nucleotide;
thermally cycling the sample between at least a denaturation temperature and an elongation temperature;
illuminating the sample with a selected wavelength of light that is absorbed by the fluorescent entity during the thermally cycling step;
monitoring an amplification dependent emission of the fluorescent entity; and
detecting the presence or absence of the variant nucleotide by judging the presence or absence of an amplified product of the DNA.

2. The method as claimed in claim 1, wherein the 3′-end nucleotide of the second primer is complementary to the 3′-end nucleotide of the first primer.

3. The method as claimed in claim 1, wherein the fluorescent entity comprises a double strand specific nucleic acid binding dye.

4. The method as claimed in claim 1, wherein the fluorescent entity comprises a fluorescently labeled oligonucleotide probe that hybridizes to the DNA.

5. The method as claimed in claim 1, wherein the variant nucleotide results from a point mutation of the target nucleic acid.

6. A kit for detecting the presence or absence of at least one variant nucleotide in a target nucleic acid contained in a sample, the kit comprising:
a pair of first and second primers that have nucleotide sequences substantially complementary to a DNA which flanks the target nucleic acid wherein the 3′-end nucleotide of the first primer is complementary to the variant nucleotide;
four different nucleoside triphosphates;
a nucleic-acid-binding fluorescent entity; and
a thermostable polymerase.

7. The kit as claimed in claim 6, wherein the 3′-end nucleotide of the second primer is complementary to the 3′-end nucleotide of the first primer.

8. The kit as claimed in claim 6, wherein the fluorescent entity comprises a double strand specific nucleic acid binding dye.

9. The kit as claimed in claim 6, wherein the fluorescent entity comprises a fluorescently labeled oligonucleotide probe that hybridizes to the DNA.

10. The kit as claimed in claim 6, wherein the variant nucleotide results from a point mutation of the target nucleic acid.
